(19) United States
(12) Patent Application Publication
(10) Pub. No.: US 2026/0000704 A1
(43) Pub. Date: Jan. 1, 2026

(54) NUTRACEUTICAL COMPOSITION OF HYALURONIC ACID

(71) Applicants: Yogesh Baburao Sutar, Jalgaon, Maharashtra (IN); Jaishree Kishor Mali, Palghar, Maharashtra (IN); Shubhangi Niket Mestry, Thane, Maharashtra (IN); Niket Kesarinath Mestry, Palghar, Maharashtra (IN)

(72) Inventors: Yogesh Baburao Sutar, Jalgaon, Maharashtra (IN); Jaishree Kishor Mali, Palghar, Maharashtra (IN); Shubhangi Niket Mestry, Thane, Maharashtra (IN); Niket Kesarinath Mestry, Palghar, Maharashtra (IN)

(21) Appl. No.: 18/869,440

(22) PCT Filed: May 26, 2023
(86) PCT No.: PCT/IN2023/050505

§ 371 Date: Nov. 26, 2024

(30) Foreign Application Priority Data
May 27, 2022 (IN) 202221004499

(51) Int. Cl. A61K 31/728; A61K 9/08; A61K 31/10; A61K 33/30; A61K 47/12; A61K 47/18

(52) U.S. Cl. CPC A61K 31/728; A61K 9/08; A61K 31/10; A61K 33/30; A61K 47/12; A61K 47/183

(57) ABSTRACT

Describe herein a nutraceutical composition containing synergistic combination of Hyaluronic acid, Dimethyl sulfone and/or zinc. Further, the invention provides a process for manufacturing the said synergistic composition.

### RELATED APPLICATIONS

The present application is the National Stage entry of International Patent Application No. PCT/IN2023/050505 filed on May 26, 2023, which is based on and claims priority to Indian Patent Application number 202221004499 filed on May 27, 2022, which are incorporated herein by reference in their entirety.

### FIELD OF INVENTION

The invention relates to a nutraceutical composition. More particularly, the present invention relates to a nutraceutical composition containing hyaluronic acid and/or salt, methylsulfonylmethane (dimethyl sulfone) and/or zinc.

### BACKGROUND OF THE INVENTION

Hyaluronic acid is a naturally occurring mucopolysaccharide generally present in all vertebrates, including humans. Hyaluronic acid (HA) is also known as hyaluronan, hylan, hyaluronate. It can include several thousand sugar molecules in the backbone to form a long chain. It is a hydrophilic molecule which binds to water to give a viscous, gelatin like molecule which hydrates, lubricates, cushions movable part of the body and hence it is also named as “nature's moisturizer” or natural humectant. Although HA was discovered in 1934 (Meyer and Palmer), it was first commercialised in 1942 by Endre Balazs as a substitute for egg white in bakery products.

Dimethyl sulfone (MSM) is natural form of bioavailable sulphur. MSM stimulates the synthesis of collagen, elastin, proteoglycans and hyaluronic acid. It is an anti-inflammatory, antioxidant, detoxifier of body and is used mostly in the treatment of arthritis, degenerative bone and joint diseases, bursitis, tendonitis, tenosynovitis, gum diseases, eye inflammation, chronic pain. Further, MSM has also shown effectiveness in clearing arteries of heart, interstitial cystitis, promotes wound healing, improves collagen production, raise energy levels, cartilage preservation, improves joint pain, treats digestive problems, improves skin health and complexion, strengthens hairs and nails, relieves allergy symptoms, boost immunity, anticancer, scleroderma, fibromyalgia, autoimmune disorders, chronic venous insufficiency, bacterial prostatitis, obesity induced metabolic disorder, reduce muscle soreness associated with exercise, rosacea, gastric ulcers, hemorrhoids etc.

Zinc is an essential trace element in human, plants, animals and microorganisms. It plays an important role in controlling diarrhea, acrodermatitis, enteropathica, cancer, diabetic neuropathy, pressure ulcer, anemia, renal disease, liver disease, eyes, skin, brain defects, sexual defects, AMD eye problem, lupus, gum health: also acts as an antiviral, antibacterial, immunomodulator, insulin stimulator, bone regenerator: prevents osteoporosis, arthritis, infectious diseases.

Therefore, it is an endeavour of the current inventors to develop a composition which contains all the three ingredients as can be seen above: all the three components have a definitive therapeutic value. However, all the three molecules have not been combined to give a composition which shows definitive synergistic action.

### SUMMARY OF INVENTION

In view of above, the current invention provides a nutraceutical composition containing synergistic combination of Hyaluronic acid, Dimethyl sulfone and/or zinc.

In another aspect, the present invention provides the process for manufacturing the said synergistic composition.

### BRIEF DESCRIPTION OF THE DRAWINGS

FIGS. 1A-1E provide a summary of the comparative observations of Example 23.

### DETAILED DESCRIPTION OF THE INVENTION

The invention will now be described in detail in connection with certain preferred and optional embodiments, so that various aspects thereof may be more fully understood and appreciated. As used herein, the following terms and phrases shall have the meaning set forth below.

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described.

Unless stated to the contrary, any of the words “contains”, “containing”, “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention.

Further, words like “a”, “an”, “at least” and “the” should be construed to not only cover singular quantities but also plural quantities of the elements immediately following them.

Described herein is a nutraceutical composition containing synergistic combination of Hyaluronic acid, Dimethyl sulfone and/or zinc. The synergistic combination of the above mentioned dietary nutrients in a single product would provide combined benefits of all in a single product. Moreover, MSM is known to stimulate the synthesis of Hyaluronic acid (HA), thus it will help in retaining the HA level throughout the body.

In accordance with the above embodiment, the composition contains Hyaluronic acid or pharmaceutical acceptable salts thereof in range of 0.5% w/v-1.5% w/v, Dimethyl sulfone (MSM) or pharmaceutical acceptable salts thereof in range of 0.5% w/v-1.5% w/v and/or zinc or pharmaceutical acceptable salts thereof in range of 0.01% w/v-0.1% w/v. Preferably the composition contains Hyaluronic acid and Dimethyl sulfone in the ratio of 1:1.

In subsequent embodiment, the composition, may further contains preservatives, selected from Potassium Sorbate, Sodium Benzoate, in range of 0.01% w/v-0.1% w/v.

In yet another subsequent embodiment, the composition of the invention preferably contains a chelating agent, such as EDTA or a salt thereof in range of 0.02% w/v-0.08% w/v.

In one of the embodiments of the invention, the vehicle for the composition is Purified water.

In one of the embodiments the invention the vehicle for the composition is 0.9% Normal Saline solution.

In another embodiment the invention provides a process for manufacturing of the composition of the invention wherein the vehicle is purified water, whereby the process comprises the following steps:
a) measuring ultrapurified water (pH 7.6-7.7) or 0.9% normal saline in a pharma grade stainless steel tank;
b) adding potassium sorbate and sodium benzoate in a tank of step a, containing water followed by stirring to dissolve till potassium sorbate and sodium benzoate are completely dissolved;
c) adding weighed amount of sodium hyaluronate in the solution of step b;
d) stirring the mixture of step c, with axial impeller (45°) at 1200 rpm for 12-15 min;
e) standing the mixture of step a, for 8 hours in a cover tank for soaking to ensure complete dissolution of sodium Hyaluronate;
f) adding weighed amount of Zinc citrate dihydrate in the mixture of step e, with stirring for 2 min at 1200 rpm;
g) adding weighed amount of Ethylenediaminetetraacetic acid disodium salt dihydrate in the mixture of step f, with stirring for 10 min at 1600 rpm, and
h) adding Dimethyl sulfone in the mixture of step g, and stir the mixture for 20 min at 1200 rpm, after complete dissolution of Zinc citrate dihydrate and Ethylenediaminetetraacetic acid disodium salt dihydrate.

### EXAMPLE

Several examples are set forth below to further illustrate the nature of the invention and the manner of carrying it out. However, the invention should not be considered as being limited to the details thereof.

### Example 1

TABLE 1
Oral liquid solution of Hyaluronic
acid (Purified water) . . . 500 mL

| Sr. | | Quantity | Percent |
|---|---|---|---|
| No | Compound | (gm) | (%) |
| 1. | Sodium hyaluronate (1.4-2.0 MDa) | 5 | 1 |
| 2. | Dimethyl sulfone | 5 | 1 |
| 3. | Zinc citrate dihydrate | 0.254 | 0.05 |
| 4. | Potassium Sorbate | 0.254 | 0.05 |
| 5. | Sodium Benzoate | 0.254 | 0.05 |
| 6. | Ethylenediaminetetraacetic | 0.375 | 0.075 |
| | acid di sodium salt dihydrate | | |
| 7. | Purified Water | 500 mL | — |

### Process for Manufacturing:

a) Measure ultrapurified water (pH 7.6-7.7) in a pharma grade stainless steel tank.
b) Add potassium sorbate and sodium benzoate in a tank containing water and stir to dissolve (stir for 2 min).
c) After complete dissolution of potassium sorbate and sodium benzoate, add weighed amount of sodium hyaluronate in it and stir the mixture with axial impeller (45°) at 1200 rpm for 12-15 min. After that cover the tank and keep the mixture overnight (8 hours) for soaking to ensure complete dissolution of sodium hyaluronate.
d) Add weighed amount of Zinc citrate dihydrate (crushed/fine) in the above mixture with stirring for 2 min at 1200 rpm.
e) Add weighed amount of Ethylenediaminetetraacetic acid disodium salt dihydrate in the above mixture with stirring for 10 min at 1600 rpm.
f) After complete dissolution of Zinc citrate dihydrate and Ethylenediaminetetraacetic acid disodium salt dihydrate (mixture will become clear), add Dimethyl sulfone in the tank containing mixture and stir the mixture for 20 min at 1200 rpm.

### Example 2

TABLE 2
Oral liquid solution of Hyaluronic acid
(0.9% saline solution) . . . 500 mL

| Sr. | | Quantity | Percent |
|---|---|---|---|
| No | Compound | (gm) | (%) |
| 1. | Sodium hyaluronate (1.4-2.0 MDa) | 5 | 1 |
| 2. | Dimethyl sulfone | 5 | 1 |
| 3. | Ethylenediaminetetraacetic | 0.125 | 0.025 |
| | acid di sodium salt dihydrate | | |
| 4. | 0.9% Sodium chloride solution | 500 mL | — |

### Procedure:

a) Measure 0.9% Sodium Chloride solution in a pharma grade stainless steel tank.
b) Add weighed amount of sodium hyaluronate in it and stir the mixture with axial impeller (45°) at 1200 rpm for 12-15 min. After that cover the tank and keep the mixture overnight (8 hours) for soaking to ensure complete dissolution of sodium hyaluronate.
c) Add weighed amount of Ethylenediaminetetraacetic acid disodium salt dihydrate in the above mixture with stirring for 10 min at 1600 rpm.
d) After complete dissolution of Ethylenediaminetetraacetic acid disodium salt dihydrate (mixture will become clear), add Dimethyl sulfone in the tank containing mixture and stir the mixture for 20 min at 1200 rpm.

### Example 3

Sodium hyaluronate—7.2 gm, MSM—7.2 gm, Sodium benzoate—0.014 gm, purified water—q.s up to 720 ml

### Procedure:

1) The weighed quantity of MSM and Sodium benzoate were dissolved in minimum amount of purified water.
2) Sodium HA was dissolved in 600 ml of water and stirred with overhead stirrer till clear solution was obtained.
3) In the next step, solution from step 1 was added to solution from step 2 and volume was made up to 720 ml with purified water.

### Observation:

The clear solution became turbid with in 24 hours.

### Example 4

Using the same method for Example 1, the following composition was prepared: Sodium hyaluronate—28.8 gm, MSM—14.4 gm, zinc citrate—0.7368 g, purified water—q.s up to 1440 ml

### Observation:

The clear solution became turbid with in 24 hours.

### Example 5

Using the same method for Example 1, the following composition was prepared, and the volume was made up to 720 ml with purified water.

Sodium hyaluronate—7.2 gm, MSM—7.2 gm, zinc citrate—0.368 gm, purified water—q.s up to 720 ml (pH of the final solution was found to be 6)

### Observation:

The clear solution became turbid with in 24 hours.

### Example 6

Sodium hyaluronate—7.2 gm, MSM—7.2 gm, zinc citrate—0.368 gm, purified water—720 ml

### Procedure:

1) The weighed quantity of Sodium HA and Zinc citrate were dissolved in 620 ml of purified water.
2) MSM was dissolved in 100 ml of water and stirred with overhead stirrer till clear solution was obtained.
3) In the next step, solution from step 1 was added to solution from step 2.
4) Final pH adjusted at 5.4 with dilute HCl.

### Observation:

The clear solution became turbid with in 24 hours.

### Example 7

Sodium hyaluronate—7.08 gm, MSM—7.08 gm, zinc citrate—0.362 gm, purified/demineralized water—708 ml

Using the same method for Example 1, the following composition was prepared, except step 4. The pH of the composition in purified water was found to be 6.5, which was adjusted at pH 5.57 with dil HCl solution. The pH of demineralized water was found to be 5.67, so it was not adjusted.

### Observation:

The clear solution became turbid after 24 hours.

### Example 8

Sodium hyaluronate—7.08 gm, MSM—7.08 gm, zinc citrate—0.362 gm, purified water—708 ml

Using the same method for Example 1, the following composition was prepared, and the pH of the final solution was 6.8 which was adjusted with citrate buffer to 5.5.

### Observation:

The clear solution became turbid after 24 hours.

### Example 9

Sodium hyaluronate—7.08 gm, MSM—7.08 gm, zinc citrate—0.362 gm, R.O. water—708 ml

Using the same method for Example 1, the following composition was prepared, and the pH of the final solution was 6.8.

### Observation:

The clear solution became turbid after 48 hours.

### Example 10

Sodium hyaluronate—7.08 gm, MSM—7.08 gm, zinc citrate—0.362 gm, R.O. water—708 ml

Using the same method for Example 1, the following composition was prepared, and the pH of the final solution was 6.8 which was adjusted with dil NaOH to 7.4.

### Observation:

The clear solution became turbid during preparation itself.

### Example 11

Sodium hyaluronate—7.08 gm, MSM—7.08 gm, zinc citrate—0.362 gm, R.O. water—q.s up to 708 ml

Using the same method for Example 1, the following composition was prepared, and the pH of the final solution was 6.8 which was adjusted with dil NaOH to 7.0.

### Observation:

The clear solution became turbid after 48 hours.

### Example 12

Sodium hyaluronate—3.54 gm, MSM—3.54 gm, zinc citrate—0.180 gm, purified water—354 ml
1) Weighed quantity of Sodium HA was dissolved in 354 ml of water, then MSM and Zinc citrate was added and stirred with overhead stirrer till clear solution was obtained.
2) Final pH of solution was 6.7 which was adjusted with dil NaOH to 7.0.

### Observation:

The clear solution became turbid within 48 hours.

### Example 13

Sodium hyaluronate—3.54 gm, MSM—3.54 gm, zinc citrate—0.180 gm, sodium benzoate—0.354 gm, purified water—354 ml

### Procedure:

1) The weighed quantity of Sodium HA and Zinc citrate were dissolved in 354 ml of purified water.
2) MSM and sodium benzoate was added to above solution and stirred with overhead stirrer till clear solution was obtained.
3) Final pH of solution was 6.76 which was adjusted with dil NaOH to 7.0.

### Observation:

The solution remained clear for 7 days.

### Example 14

Sodium hyaluronate—7.08 gm, MSM—7.08 gm, zinc citrate—0.362 gm, sodium benzoate—0.354 gm R.O. water (filtered through 0.45 μm membrane filter)—708 ml

### Procedure:

1) The weighed quantity of Sodium HA was dissolved in 708 ml of R.O. water then Zinc citrate was added in it.
2) MSM and sodium benzoate was added to the above solution and stirred with overhead stirrer till clear solution was obtained.
3) Final pH of solution was 7 which was not adjusted.

### Observation:

The clear solution became turbid within 28 hours.

### Example 15

Sodium hyaluronate—7.08 gm, MSM—7.08 gm, zinc citrate—0.362 gm, sodium benzoate—0.354 gm R.O. water (filtered through 0.45 μm membrane filter)—708 ml

### Procedure:

1) The weighed quantity of Sodium HA was dissolved in 708 ml of R.O. water then Zinc citrate was added in it.
2) MSM and sodium benzoate was added to the above solution and stirred with overhead stirrer till clear solution was obtained.
3) Final pH of solution was 7 which was adjusted with citrate buffer (pH-3.75) to 5.5.

### Observation:

The clear solution became turbid within 4 days.

### Example 16

Sodium hyaluronate—7.08 gm, MSM—7.08 gm, zinc citrate—0.362 gm, sodium benzoate—0.354 gm R.O. water (filtered through 0.45 μm membrane filter)—708 ml

### Procedure:

1) The weighed quantity of Sodium HA was dissolved in 708 ml of R.O. water then Zinc citrate was added in it.
2) MSM and sodium benzoate was added to the above solution and stirred with overhead stirrer till clear solution was obtained.
3) Final pH of solution was 7 which was adjusted with citrate buffer (pH-3.75) to 6.6.

### Observation:

The clear solution became turbid within 48 hour.

### Example 17

Sodium hyaluronate—5 gm, MSM—5 gm, zinc citrate—0.254 gm, sodium benzoate—0.25 gm, potassium sorbate—0.25 gm, purified water—500 ml

### Procedure:

1) The weighed quantity of sodium benzoate and potassium sorbate were dissolved in 500 ml purified water.
2) Then sodium HA was added till clear solution was obtained. Subsequently, zinc citrate was added followed by MSM to the above solution and stirred with overhead stirrer till clear solution was obtained.
3) Final pH of solution was 6.9 which was adjusted with citrate buffer (pH-3.75) to 5.

### Observation:

The clear solution became turbid after 4 days.

### Example 18

Sodium hyaluronate—5 gm, MSM—5 gm, zinc citrate—0.254 gm, potassium sorbate—0.5 gm, purified water—500 ml

### Procedure:

1) The weighed quantity of potassium sorbate was dissolved in 500 ml purified water.
2) Then sodium HA was added till clear solution was obtained. Subsequently, zinc citrate was added followed by MSM to the above solution and stirred with overhead stirrer till clear solution was obtained.
3) Final pH of solution was 6.9 which was adjusted with citrate buffer (pH-3.75) to 5.

### Observation:

The clear solution became turbid after 7 days.

### Example 19

Sodium hyaluronate—5 gm, MSM—5 gm, zinc citrate—0.254 gm, Disodium EDTA—0.254 gm, sodium metabisulphite—0.5 gm, potassium sorbate—0.5 gm, purified water—500 ml

### Procedure:

1) The weighed quantity of potassium sorbate was dissolved in 500 ml purified water.
2) Then sodium HA was added till clear solution was obtained. Subsequently, zinc citrate, Disodium EDTA and sodium metabisulphite was added followed by MSM to the above solution and stirred with overhead stirrer till clear solution was obtained.
3) pH of the final solution was adjusted with citrate buffer (pH-3.75) to 5.

### Observation:

The viscosity of the composition was decreased after 15 days and it became turbid after 20 days.

### Example 20

Sodium hyaluronate—5 gm, MSM—5 gm, zinc citrate—0.254 gm, sodium metabisulphite—0.254 gm, Disodium EDTA—0.254 gm, sodium benzoate—0.5 gm, purified water—500 ml

### Procedure:

1) The weighed quantity of sodium benzoate was dissolved in 500 ml purified water.
2) Then sodium HA was added till clear solution was obtained. Subsequently, zinc citrate, Disodium EDTA and sodium metabisulphite was added followed by MSM to the above solution and stirred with overhead stirrer till clear solution was obtained.
3) pH of the final solution was 4.8 which was adjusted with dil. NaOH to 5.

### Observation:

The viscosity of the composition was decreased after 15 days and it became turbid after 20 days.

### Example 21

Sodium hyaluronate—5 gm, MSM—5 gm, zinc citrate—0.254 gm, Disodium EDTA—0.254 gm, potassium sorbate—0.5 gm, purified water—500 ml

### Procedure:

1) The weighed quantity of potassium sorbate was dissolved in 500 ml purified water.
2) Then sodium HA was added till clear solution was obtained. Subsequently, zinc citrate, Disodium EDTA was added followed by MSM to the above solution and stirred with overhead stirrer till clear solution was obtained.
3) pH of the final solution was adjusted with citrate buffer to 5.

### Observation:

The clear solution became turbid after 15 days.

### Example 22

Sodium hyaluronate—5 gm, MSM—5 gm, zinc citrate—0.254 gm, sodium benzoate—0.254 gm, potassium sorbate—0.254 gm, purified water—500 ml

### Procedure:

1) The weighed quantity of sodium benzoate and potassium sorbate were dissolved in 500 ml of purified water.
2) Then sodium HA was added till clear solution was obtained. Subsequently, zinc citrate was added followed by MSM to the above solution and stirred with overhead stirrer till clear solution was obtained.
3) pH of the final solution was adjusted with citrate buffer to 5.

### Observation:

The batch remained stable for almost 6 months after that it became turbid.

### Example 23

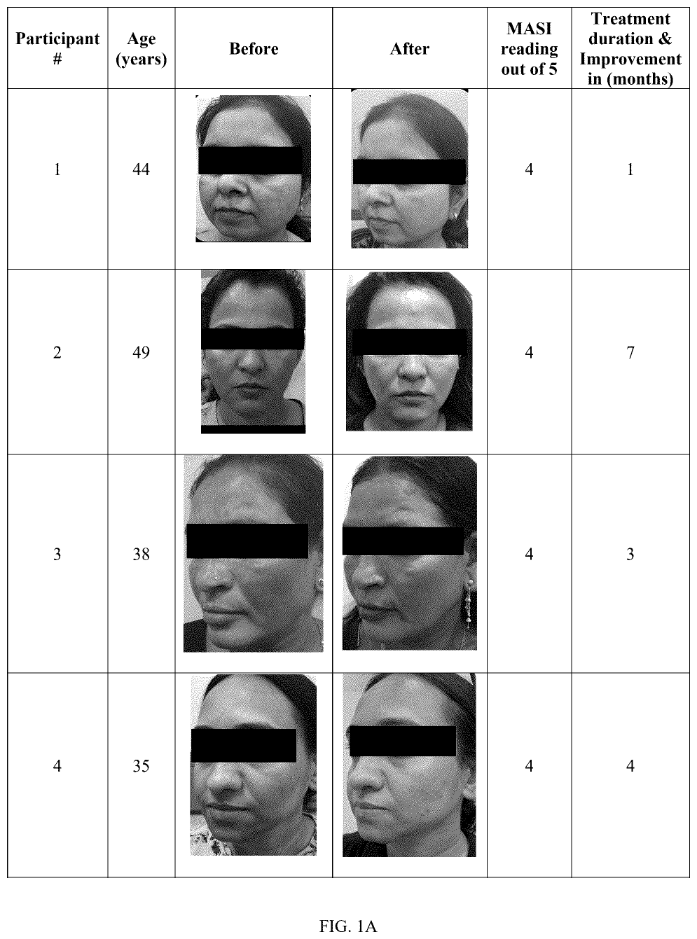

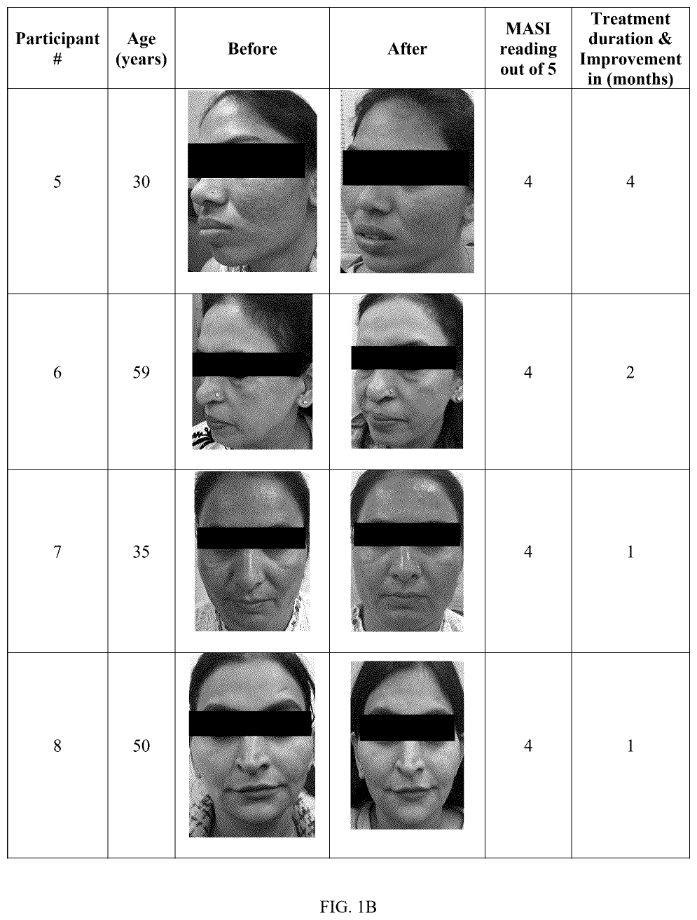

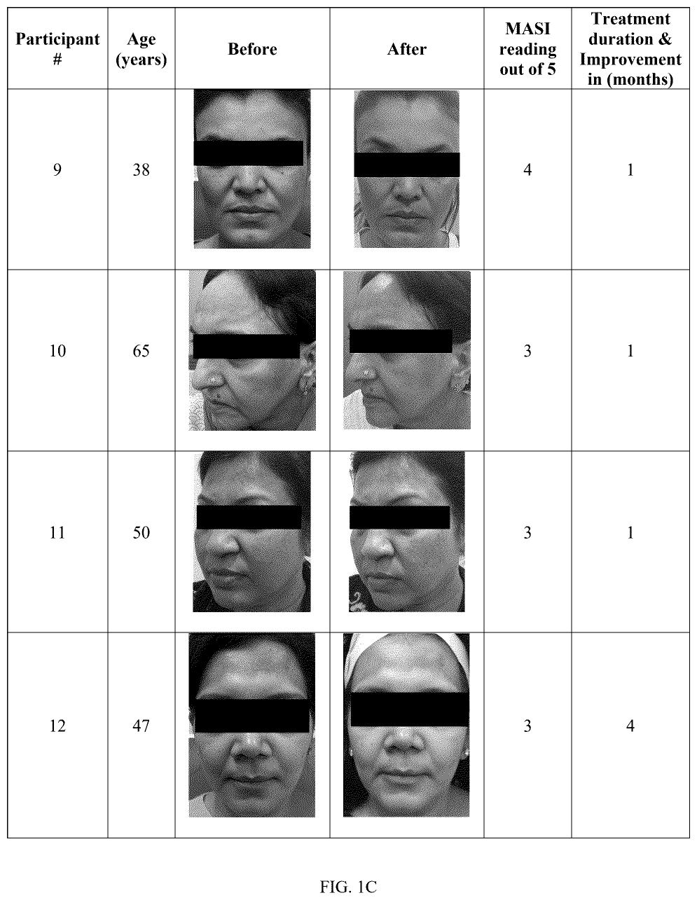

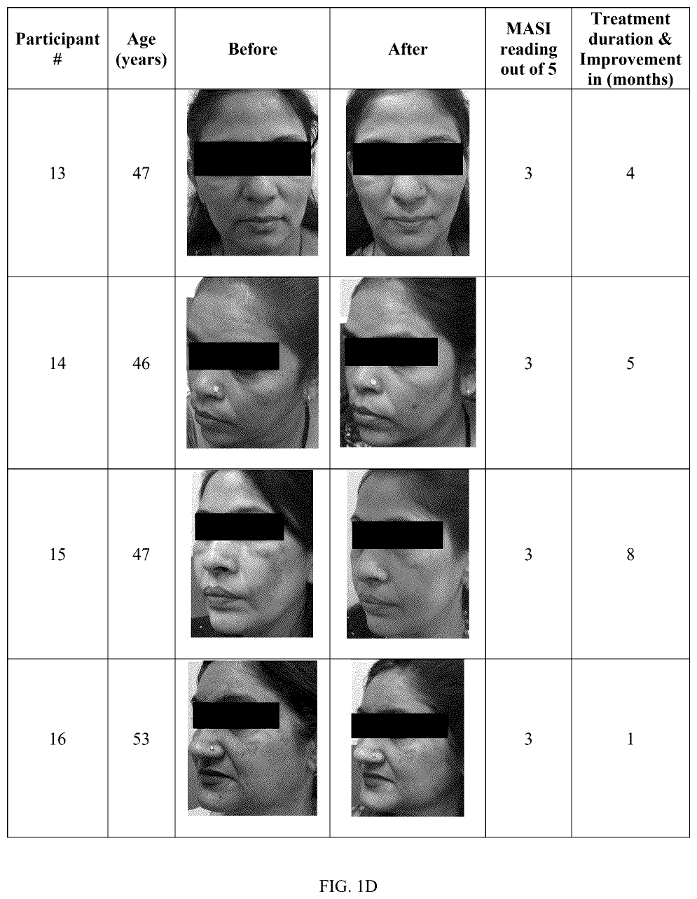

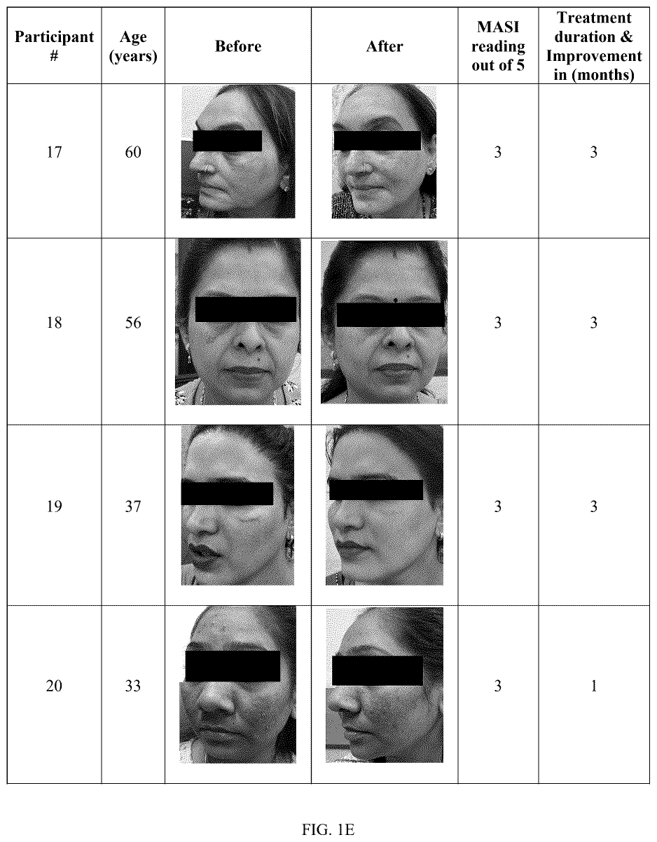

20 females with moderate to severe melasma conditions were included as participants. All participants were treated with the same dose (6 mL) of oral hyaluronic acid solution (purified/saline) with variable duration. Around 45% of participants showed a very good response (MASI 4 out of 5) for the novel oral hyaluronic acid solution. While 55% of participants showed a good response (MASI 3 out of 5). The average duration of treatment for all the participants was approximately 4 weeks. The comparative observations are presented in FIGS. 1A-1E.

1. A nutraceutical composition comprising:
a) hyaluronic acid or a salt thereof,
b) methylsulfonylmethane (dimethyl sulfone) or a salts thereof, and
c) zinc or a pharmaceutically acceptable salts thereof,
wherein the nutraceutical composition provides combined benefits of all the components in a single composition.

2. The nutraceutical composition as claimed in claim 1, wherein the composition comprises:
hyaluronic acid or a pharmaceutical acceptable salts thereof in a range of 0.5% w/v-1.5% w/v;
dimethyl sulfone (MSM) or a pharmaceutical acceptable salts thereof in a range of 0.5% w/v-1.5% w/v, and
zinc or a pharmaceutical acceptable salts thereof in a range of 0.01% w/v-0.1% w/v.

3. The nutraceutical composition as claimed in claim 1, wherein the composition contains hyaluronic acid and dimethyl sulfone in a ratio of 1:1.

4. The nutraceutical composition as claimed in claim 1, wherein the composition further contains a preservatives, selected from potassium sorbate, and sodium benzoate.

5. The nutraceutical composition as claimed in claim 1, wherein the composition contains a chelating agent, selected from ethylenediaminetetraacetic acid or a salt thereof.

6. The nutraceutical composition as claimed in claim 1, wherein the composition contains purified water as a vehicle.

7. The nutraceutical composition as claimed in claim 1, wherein the composition contains 0.9% normal saline solution as a vehicle.

8. The nutraceutical composition as claimed in claim 1, wherein the pharmaceutical acceptable salt of hyaluronic acid is sodium hyaluronate.

9. The nutraceutical composition as claimed in claim 1, wherein the zinc is in a form of zinc citrate.

10. The process for manufacturing the nutraceutical composition as claimed in claim 1, wherein the process comprises following steps:
a) measuring ultrapurified water (pH 7.6-7.7) or 0.9% normal saline in a pharma grade stainless steel tank;
b) adding potassium sorbate and sodium benzoate in a tank of step a), containing water followed by stirring to dissolve until the potassium sorbate and the sodium benzoate are completely dissolved;
c) adding a weighed amount of sodium hyaluronate in the solution of step b);
d) stirring the mixture of step c), with an axial impeller (45°) at 1200 rpm for 12-15 min;
e) standing the mixture of step a), for 8 hours in a cover tank for soaking to ensure complete dissolution of sodium hyaluronate;
f) adding a weighed amount of zinc citrate dihydrate in the mixture of step e), with stirring for 2 min at 1200 rpm;
g) adding a weighed amount of ethylenediaminetetraacetic acid disodium salt dihydrate in the mixture of step f), with stirring for 10 min at 1600 rpm; and
h) adding dimethyl sulfone in the mixture of step g), and stirring the mixture for 20 min at 1200 rpm, after complete dissolution of zinc citrate dihydrate and ethylenediaminetetraacetic acid disodium salt dihydrate.

11. The nutraceutical composition as claimed in claim 4, wherein the preservative is present in a range of 0.01% w/v-0.1% w/v.

12. The nutraceutical composition as claimed in claim 5, wherein the chelating agent is present in a range of 0.02% w/v-0.08% w/v.

13. The nutraceutical composition as claimed in claim 2, wherein the pharmaceutical acceptable salt of hyaluronic acid is sodium hyaluronate.

14. The nutraceutical composition as claimed in claim 2, wherein the zinc is in a form of zinc citrate.
